(19) United States
(12) Patent Application Publication
(10) Pub. No.: US 2002/0099429 A1
(43) Pub. Date: Jul. 25, 2002

(54) Xylite crystals for use as cooling means for protection of a severed or injured body part of a human or animal

(76) Inventor: Gunter H. Marx, Gauting (DE)

Correspondence Address: HENRY M FEIEREISEN, 350 FIFTH AVENUE, SUITE 3220, NEW YORK, NY, 10118, US

(21) Appl. No.: 09/765,810

(22) Filed: Jan. 19, 2001

(51) Int. Cl. A61F 7/00

(52) U.S. Cl. 607/108; 607/114

(57) ABSTRACT

The use of xylite for use as a cooling means, wherein xylite is in a crystalline form having crystals only at a grain size from 0.2 mm to 1.5 mm.

### BACKGROUND OF THE INVENTION

The present invention relates, in general, to the use of xylite crystals as cooling means for protection of a severed or injured body part of a human or animal.

In medical practice, it is generally desired to cool injured parts below the usual body temperature as quickly as possible after the injury has occurred, in order to slow down the metabolism in the cells in the region of the injury until treatment can be applied, and to eliminate reactions which are disadvantageous in regard to the heating process. Thus it is known for the region of the body affected by injury to be cooled with iced water in the case of sprains or ligament strains but also in the case of open wounds. Further, it is known for example for parts of a human body which have been severed in an accident to be kept at the lowest possible temperature above freezing point until surgery (replantation) can be performed. Body parts of living beings should however be protected against injury from frost, and the cooling of a body part should never proximate the freezing point of blood at −0.5° C. Desired is a cooling of a body part to a temperature between +2° C. and +8° C.

In many cases, it is not possible to effectively cool the injured body part as quickly as possible after an injury has been suffered, due to the absence of a suitable cooling agent. This frequently applies even in regard to first-aid stations, ambulances and the like as ice for making iced water is available only when it can be constantly stored independently of the outside temperature. A concern in particular in cases a limb becomes severed, is the fear of danger of infection or other damage caused by the cooling agent. Therefore, open injuries, for example open fractures of limbs, cannot oftentimes not be cooled by means of water which possibly comes directly into contact with the wound.

There is therefore a need for a cooling means or agent for cooling injured parts or areas of a body to be held in preparation, at least in accident stations, ambulance vehicles and the like, which cooling means or agent can be stored unrestrictedly and irrespective of the outside temperature without separate cooling arrangements, and which does not give rise to any danger of infection or poisoning even directly contacting injured or damaged tissue. Cooling packs have been known, which use an endothermic solution reaction of a salt in water for cooling surrounding regions, for example ammonium nitrate which removes heat from the environment when it is dissolved in water and thereby causes a substantial drop in temperature.

However, cooling agents of this kind are inconceivable for use in a medical connection for the above-discussed cooling purposes because, like ammonium nitrate, they are toxic and corrosive or caustic so that considerable damage is encountered when directly contacting a wound. Although protections were proposed to avoid a direct contact of cooling agents with injured body parts, for example, by suitable packaging the cooling agents, the risk is still too high because of a danger that such packaging may become leaky or damaged.

German Pat. No. 29 49 909 describes a container for transporting severed body parts, with the container including an inner bag for receiving the body part and an outer bag for receiving a cooling agent such as water and ice cubes. U.S. Pat. No. 4,427,010 describes a cooling pack which contains in separate compartments xylite (C₅H₁₂O₅) in crystalline form and an aqueous or non-aqueous fluid. Upon use, the xylite is dissolved in the liquid, and subsequently the resultant solution is applied in direct or indirect contact with the body part to be cooled. A problem associated with this type of cooling pack is the possibility that the body part becomes excessively cooled, in particular when outside temperatures are fairly low, e.g. during the winter season.

It would be desirable and advantageous to provide a cooling agent that obviates problems associated with conventional cooling devices and effectively cools down a body part, without the risk of frostbites

### SUMMARY OF THE INVENTION

The present invention provides for the use of xylite for application as a cooling means, with the xylite in crystalline form having crystals of a grain size between a minimum of 0.2 mm and a maximum of 1.5 mm only, for subsequent dissolution in water.

Xylite (wood sugar) is a known sugar substitute which at ambient temperature of e.g. between −10° C. and +50° C. may be present in crystalline form or also in amorphous form. Xylite is completely non-toxic and harmless to the human body, both in the dissolved and in the crystalline state, and can be used with a large number of conventional and in particular transparent or translucent packaging materials so that, in indirect cooling (cooling by heat transfer by way of an interposed container wall), the position of a cooling device relative to the body part to be cooled can be monitored and controlled.

It has now been found that the grain size of xylite crystals directly correlates with the temperature that can be reached when dissolving the xylite crystals in water. In general, through inoculation amorphous xylite with crystals, energy is released that results ultimately in a general crystallization, releasing heat of about 95° C. This process can be reversed by dissolving the thus-obtained xylite crystals in water so as to return xylite back to the amorphous state, thereby consuming the same heat as previously gained during crystallization. The heat required for transforming xylite crystals to the amorphous state is drawn from the water.

As has been surprisingly found, when dissolving xylite crystals of a grain size from 0.2 to 1.5 mm in water only, a cooling effect can be realized that will not drop below a temperature of about +5° C., so that a sufficient cooling of a body part is realized and the risk of injury as a result of excess cooling is eliminated. The reason for this is as follows: As water cools down during change of the xylite crystals to the amorphous state and reaches a temperature of e.g. +5° C., there is only a very limited amount of heat available in the water. This limited amount of heat is insufficient for xylite crystals to transform to the amorphous state so that the dissolution of the xylite crystals stops. Undissolved xylite crystals deposit as residue on the bottom of a container, much like sugar would deposit in a cup of tea when added in excessive amounts.

Dissolution of such xylite crystals in water should be executed without agitation through use of electrical or mechanical stirrers, because the resultant intimate contact between the xylite crystals and water may lead to a further drop of the temperature. Still, also in this case, the temperature will not fall below +2° C.

The use of xylite in a manner according to the present invention has another advantage. During transport of limbs, severed as a result of an accident, from the site of accident to the hospital, external heat, e.g. during summer, raises the temperature of the solution of xylite in water, so that this heat results in an additional dissolution of xylite crystals. The dissolution continues until saturation occurs at approximately 64% by weight, i.e. 64 gram of xylite and 36 gram of water. At this point, no further dissolution of xylite crystals takes place. Thus, during transport to the hospital, there is no need to add further xylite when initially including an excessive amount of xylite because the system will always dissolve so much xylite crystals that a temperature of about +5° C. is maintained, so long as xylite can be dissolved. This process resembles, in effect, the operation of a thermostat.

Another aspect of the present invention provides for a container for cooling a body part of a human or animal, with the container including an inner bag intended to receive a body part, and an outer bag substantially embracing the inner bag and intended to receive xylite in crystalline form having crystals at a grain size from 0.2 mm to 1.5 mm only. Upon use of the container and after placing a severed body part inside the inner bag, the outer bag is filled with xylite crystals at a grain size from 0.2 mm to 1.5 mm only and water, thereby dissolving the xylite crystals so that the contained water and ultimately the severed body part are cooled down.

Another aspect of the present invention provides for a cooling device which includes a container having two compartments separated from one another, with one of the compartments containing water, and the other one of the compartments containing xylite in crystalline form having crystals at a grain size from 0.2 mm to 1.5 mm only. After rupturing the partition between the two compartments, xylite and water are united for dissolution of the xylite crystals.

### BRIEF DESCRIPTION OF THE DRAWING

The above and other objects, features and advantages of the present invention will be more readily apparent upon reading the following description of a preferred exemplified embodiment of the invention with reference to the accompanying drawing, in which:

FIG. 1 is a schematic illustration of a conventional container for use with a cooling means according to the present invention; and

FIG. 2 is a schematic illustration of another exemplified container for use with a cooling means according to the present invention.

### DESCRIPTION OF A PREFERRED EMBODIMENT

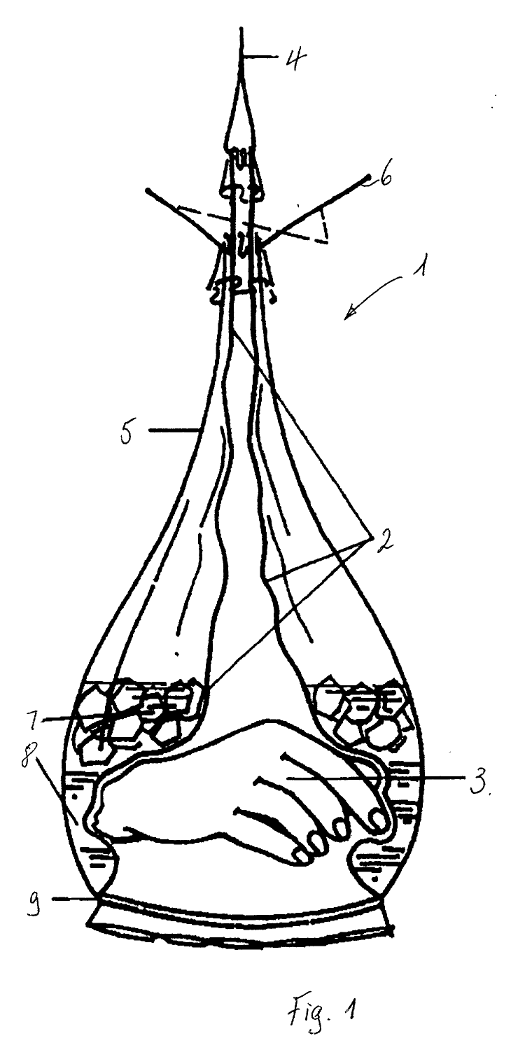

Turning now to the drawing, and in particular to FIG. 1, there is own a schematic illustration of a conventional container for use with a cooling means according to the present invention, generally designated by reference numeral 1. The container 1 includes an inner bag 2 which is open at the top for allowing placement of a severed body part, e.g. a hand 3. A thread 4 is provided to tie and thereby close the upper end of the inner bag 2. Substantially surrounding the inner bag 2 is an outer bag 5 which is open at the top and provided for introduction of xylite crystals 7 of a grain size from a minimum of 0.2 mm to a maximum of 1.5 mm only, and water 8. A thread 6 is provided to tie and thereby close the upper end of the outer bag 5. At their lower end, the inner bag 2 and the outer bag 5 are interconnected by a welding seam 9.

The container 1 is used as follows: A severed body part (e.g. hand 3) is placed into the inner bag 2, and the bag 2 is closed by tightening the thread 4. The outer bag 5 is then filled with xylite crystals of a grain size from 0.2 mm to 1.5 mm only, and water. Preferably, equal amounts of xylite crystals 7 and water 8 are introduced. The outer bag 5 is then closed. The xylite crystals 7 draw hereby heat from the water 8 and convert into the amorphous state, thereby cooling down the water and ultimately the severed body part 3.

The xylite crystals, involved here, are suitably stored in a separate package that is impervious to moisture and made e.g. of aluminum to keep the xylite crystals dry until ready for use.

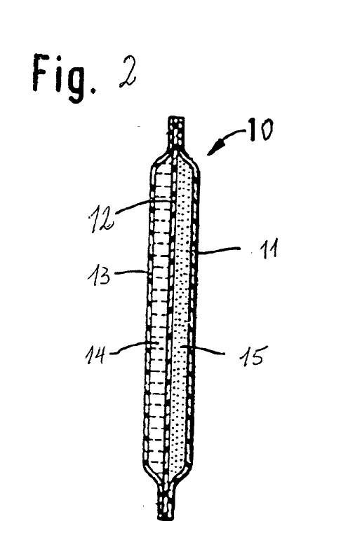

Turning now to FIG. 2, there is shown a sectional view of another exemplified container for use with a cooling means according to the present invention, generally designated by reference numeral 10. The container 10 may be made of plastic material, e.g. polyurethane, and includes three spaced-apart sheets 11, 12 and 13 suitably welded together along their edges to form two compartments 14, 15 separated by the middle sheet 12. The compartment 14 contains only germ-free water, and the compartment 15 contains xylite in crystalline form, whereby the crystals of the xylite have a grain size from a minimum of 0.2 mm to a maximum of 1.5 mm only.

Suitably, the partition sheet 12 is formed with a weakened portion (not shown) over its width, to facilitate a destruction of the partition sheet 12, e.g. by applying a longitudinal pulling force or pressurizing the contents in the container 10, to thereby unite the xylite crystals in compartment 15 with the water in compartment 14 and dissolve the xylite crystals in water. The container 10 can then be applied to the body part (not shown here) to be cooled. A tape or bandage (not shown) may be used to secure the container 10 in place.

While the invention has been illustrated and described as embodied in a container, it is not intended to be limited to the details shown since various modifications may be made without departing in any way from the spirit of the present invention.

What is claimed as new and desired to be protected by Letters Patent is set forth in the appended claims:

What is claimed is:

1. A method of cooling a body part of a human or animal, comprising the steps of:
dissolving in water xylite in crystalline form having crystals at a grain size from 0.2 mm to 1.5 mm only; and
applying the resultant solution in direct or indirect contact with a body part.

2. A cooling device for cooling a body part of a human or animal, comprising xylite in crystalline form having crystals at a grain size from 0.2 mm to 1.5 mm only.

3. A container for cooling a body part of a human or animal, said container comprising an inner bag receiving a body part, and an outer bag substantially embracing the inner bag and receiving xylite in crystalline form having crystals at a grain size from 0.2 mm to 1.5 mm only.

4. The container of claim 3 wherein water is added to the outer bag for dissolution of the xylite crystals.

5. A cooling device, comprising a container having two compartments separated from one another, one of the compartments containing water, and the other one of the compartments containing xylite in crystalline form having crystals at a grain size from 0.2 mm to 1.5 mm only.

6. The use of xylite for application as a cooling means, wherein xylite is in a crystalline form having crystals at a grain size from 0.2 mm to 1.5 mm only.

7. A solution for use as cooling means for a body part of a human or animal, comprising an effective amount of water; and xylite in crystalline form having crystals at a grain size from 0.2 mm to 1.5 mm only.

8. A cooling composition, comprising xylite in a crystalline form having crystals at a grain size from 0.2 mm to 1.5 mm only.

9. The cooling composition of claim 8, wherein the xylite crystals are stored in a package impervious to moisture.

10. The cooling composition of claim 9, wherein the package is made of aluminum.
